(19) United States
(12) Patent Application Publication
(10) Pub. No.: US 2026/0116895 A1
(43) Pub. Date: Apr. 30, 2026

(54) CRYSTAL AND METHOD FOR PRODUCING THE SAME

(71) Applicant: SETOLAS Holdings, Inc., Takamatsu-shi (JP)

(72) Inventors: Satoshi NOMURA, Takamatsu-shi (JP); Junichiro WAKAMATSU, Takamatsu-shi (JP)

(73) Assignee: SETOLAS Holdings, Inc., Takamatsu-shi (JP)

(21) Appl. No.: 19/343,604

(22) Filed: Sep. 29, 2025

(30) Foreign Application Priority Data
Sep. 30, 2024 (JP) 2024-171335
Sep. 30, 2024 (JP) 2024-171458

(51) Int. Cl. C07F 3/02; A61K 9/20; A61K 31/198

(52) U.S. Cl. CPC C07F 3/02; A61K 9/2009; A61K 9/2013; A61K 9/2054; A61K 31/198; C07B 2200/13

(57) ABSTRACT

[Problems to be solved] An object of the present disclosure is to provide magnesium glycinate having a high magnesium concentration.

[Solutions to the Problems] Magnesium glycinate having, in a powder X-ray diffraction spectrum, a first peak having a top in a 2θ range of 11.1° or more and 12.1° or less; and a second peak having a top in a 2θ range of 13.30 or more and 14.3° or less.

### TECHNICAL FIELD

The present disclosure relates to a crystal and a method for producing the same.

### BACKGROUND ART

Magnesium glycinate is superior in bioabsorbability and has weak in side reactions such as a laxative action, and thus can be used, for example, for food and drink products, dietary supplements, pharmaceutical products.

Patent Document 1 describes a magnesium glycinate hydrate having a structure represented by the following formula.

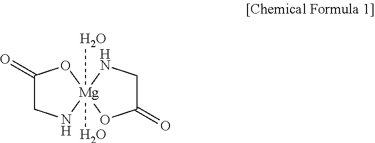

Patent Document 2 describes a method of obtaining an insoluble metal organic chelate by adding an organic acid ligand and a metal compound to a non-aqueous liquid to form a suspension, allowing the organic acid ligand and the metal compound to react, heating the suspension with stirring for a prescribed time, and filtering the suspension.

Patent Document 3 describes a method of triggering a solid body reaction for forming a metal chelate by exposing a solvent-free mixture of a metal compound and a solid organic acid containing a chelate-forming acid such as α- and β-amino acids or hydroxycarboxylic acids to strong mechanical stress.

### PRIOR ART

### Patent Documents

Patent document 1: US 2014/0186408 A
Patent document 2: JP 2005-145822 A
Patent document 3: JP 2020-517602 A

### SUMMARY

### Problems to be Solved

In the magnesium glycinate hydrate described in Patent Document 1 and the magnesium glycinate compounds obtained by the production methods described in Patent Documents 1 and 2, the magnesium concentration is not sufficiently satisfactory.

An object of the present disclosure is to provide magnesium glycinate having a high magnesium concentration.

### Solutions to the Problems

According to a first embodiment of the present disclosure, provided is a magnesium glycinate having a first peak having a top in a 2θ range of 11.1° to 12.1°; and a second peak having a top in a 2θ range of 13.30 to 14.3° in a powder X-ray diffraction spectrum.

In a second embodiment of the present disclosure, in the magnesium glycinate of the first embodiment, a ratio I₁/I₂ of an intensity I₁ of the first peak to an intensity I₂ of the second peak may be 2 to 2.5.

In a third embodiment of the present disclosure, the magnesium glycinate according to one of the first and second embodiments may further have, in the powder X-ray diffraction spectrum, one or more peaks selected from a third peak having a top in a 2θ range of 19.5° to 20.5°; and a fourth peak having a top in a 2θ range of 37.5° to 38.5°.

In a fourth embodiment of the present disclosure, the magnesium glycinate according to any one of the first to third embodiments may have, in the powder X-ray diffraction spectrum, one or more peaks selected from among a fifth peak having a top in a 2θ range of 17.4° to 18.4°; a sixth peak having a top in a 2θ range of 18.1° to 19.1°; a seventh peak having a top in a 2θ range of 22.9° to 23.9°; and an eighth peak having a top in a 2θ range of 28.6° to 29.6°.

A fifth embodiment of the present disclosure provides magnesium glycinate in which a rate of weight loss at 380° C. based on the weight at 50° C. is 30 wt % or less in thermogravimetric analysis.

In the fifth embodiment of the present disclosure, in the magnesium glycinate according to any one of the first to fourth embodiments, a rate of weight loss at 380° C. based on the weight at 50° C. may be 30 wt % or less.

In a sixth embodiment of the present disclosure, in any one of the magnesium glycinates according to the first to fifth embodiments, a value S₁/S₂ obtained by dividing an area S₁ of an endothermic peak having an onset temperature in a range of 125° C. to 160° C. by an area S₂ of an endothermic peak having an onset temperature in a range of 330° C. to 370° C. may be 0 to 1.8.

In a seventh embodiment of the present disclosure, in any one of the first to sixth embodiments, the magnesium glycinate may be free of hydration water.

In the eighth embodiment of the present disclosure, in any one of the first to seventh embodiments described, the color difference ΔE* may be 30 or less. The color difference may be measured by the following method.

### Method for Measuring Color Difference

A mixture is obtained by mixing 1462 parts by mass of glucose and 500 parts by mass of magnesium glycinate and storing the mixture at 60° C. for 72 hours under a sealed condition. A color difference, ΔE*, relative to a reference point (L₀*=100, a₀*=0, b₀*=0) in an L*a*b* color system at a 10° viewing angle under D65 light source is calculated using an L* value, L*₁, an a* value, a*₁, and a b*value b*₁, of the mixture, according to the following formula.

$$Δ {E}^{*} = {[ {( {L}_{1}^{*} - {L}_{0}^{*} )}^{2} + {( {a}_{1}^{*} - {a}_{0}^{*} )}^{2} + ( {b}_{1}^{*} - {b}_{0}^{*} ) 2 ]}^{1 / 2}$$

In the ninth embodiment of the present disclosure, in any one of the first to eighth embodiments described, the tensile strength of the tablet obtained from magnesium glycinate may be 75 N/cm² to 500 N/cm². The magnesium glycinate tablet may be obtained by compressing at a compression pressure of 5 kN per tablet. The above compression may be performed using a flat punch.

According to the tenth embodiment of the present disclosure, provided is a magnesium glycinate tablet comprising magnesium glycinate according to any one of the first through ninth embodiments.

According to the eleventh embodiment of the present disclosure, provided is a food and drink product or a pharmaceutical product comprising the magnesium glycinate according to any one of the first to ninth embodiments.

A twelfth embodiment of the present disclosure provides a method for producing a food and drink product or a pharmaceutical product using the magnesium glycinate according to any one of the first to ninth embodiments.

A tenth embodiment of the present disclosure provides a method for producing magnesium glycinate.

This production method comprises a first step of mixing glycine and magnesium hydroxide in the presence of water to obtain a mixture.

This production method comprises a second step of further mixing the mixture with ethanol to obtain magnesium glycinate.

In an eleventh embodiment of the present disclosure, the first step in the tenth embodiment may be performed at a temperature of 50° C. or less.

### Effects of the Invention

According to the present disclosure, magnesium glycinate having a high magnesium concentration can be provided.

### BRIEF DESCRIPTION OF DRAWINGS

FIG. 1 is a powder X-ray diffraction spectrum of Test Example 8;

FIG. 2 is a powder X-ray diffraction spectrum of Test Example 1;

FIG. 3 is a powder X-ray diffraction spectrum of Test Example 4;

FIG. 4 shows powder X-ray diffraction spectra of (a) Test Example 2, (b) Test Example 3, (c) Test Example 5, and (d) Test Example 6;

FIG. 5 shows powder X-ray diffraction spectra of (a) Test Example 7, (b) Test Example 9, (c) Test Example 10, and (d) Test Example 11;

FIG. 6 shows powder X-ray diffraction spectra of (a) Test Example 12, (b) Test Example 13, and (c) Test Example 14;

FIG. 7 is a thermogravimetric-differential scanning calorimetric curve of Test Example 8;

FIG. 8 is a thermogravimetric-differential scanning calorimetric curve of Test Example 4;

FIG. 9 is a thermogravimetric-differential scanning calorimetric curve of Test Example 10;

FIG. 10 shows thermogravimetric-differential scanning calorimetric curves of (a) Test Example 6, (b) Test Example 9, and (c) Test Example 12;

FIG. 11 shows mass spectrometry spectra of (a) Test Example 8 and (b) Test Example 4; and

FIG. 12 is a graph showing the results of the hygroscopicity tests of Test Examples 8, 1, and 4.

### DETAILED DESCRIPTION

The present disclosure relates to magnesium glycinate. In the present specification, numerical ranges each mean a range including the upper and lower limit values unless otherwise specified.

Magnesium glycinate as a compound is considered to have a structure in which two ligands having a structure that one hydrogen atom is eliminated from glycine are bonded to one magnesium atom. Magnesium glycinate may be present as an amorphous solid or as a crystal. The crystal may comprise hydration water. In the present disclosure, a six-coordination crystal containing hydration water may be referred to as type I, and a four-coordination crystal free of hydration water may be referred to as type II.

In the present disclosure, when simply referred to as magnesium glycinate, the magnesium glycinate is not limited to magnesium glycinate as a compound, and comprises a material comprising a component other than magnesium glycinate. In such a material, the form of magnesium glycinate is not particularly limited, and may be either a crystal or an amorphous solid.

In the present disclosure, a crystal means a substance in which a peak is observed when X-ray diffraction measurement is performed. In the present disclosure, the crystal of magnesium glycinate may be either a single crystal or a polycrystal of magnesium glycinate. In the present disclosure, when referring to a crystal of magnesium glycinate, the crystal is not limited to one composed only of magnesium glycinate, and also comprises one comprising a substance other than magnesium glycinate.

### The First Embodiment: Magnesium Glycinate

The magnesium glycinate of the present disclosure has, in a powder X-ray diffraction (XRD) spectrum, a first peak having a top in a 2θ range of 11.10 to 12.10 and; a second peak having a top in a 2θ range of 13.30 to 14.3°. In a preferred embodiment, the first peak may be a peak having the highest intensity in a 2θ range of 5° to 70°, and the second peak may be a peak having the second highest intensity in a 20 range of 5° to 70°.

The present disclosure provides a magnesium glycinate having a high magnesium concentration. As a result, the magnesium glycinate of the present disclosure is expected to exhibit high bioabsorbability. The present disclosure should not be construed as being limited to a particular theory, but the reason the magnesium glycinate of the present disclosure exhibits the effect is considered as follows.

That is, the magnesium glycinate of the present disclosure has the first peak and the second peak in the XRD spectrum, and it is considered to have a crystal structure derived from these peaks. In such a crystal structure, the amount of hydration water is reduced, and as a result, magnesium glycinate having a high magnesium concentration is considered to be obtained. As shown in FIG. 1 of Patent Document 1, for example, conventionally known magnesium glycinate has a peak with the highest intensity in the 2θ range of 160 to 170 in an XRD spectrum, and is considered to have a different structure as a crystal.

The top of the first peak is preferably in a 2θ range of 11.30 to 11.9°, and more preferably in a 2θ range of 11.50 to 11.7° in a powder X-ray diffraction (XRD) spectrum. The top of the second peak is preferably in a 2θ range of 13.5° to 14.1°, and more preferably in a 2θ range of 13.70 to 13.9° in a powder X-ray diffraction (XRD) spectrum.

The ratio I₁/I₂ of the intensity I₁ of the first peak to the intensity I₂ of the second peak may be preferably 2 to 2.5, and more preferably 2 to 2.3. When the intensity ratio I₁/I₂ is in such a range, the magnesium concentration in magnesium glycinate can be further increased.

In a preferred embodiment, the magnesium glycinate of the present disclosure preferably further has, in an XRD spectrum, at least one peak selected from a third peak having a top in a 2θ range of 19.5° to 20.5°; and a fourth peak having a top in a 2θ range of 37.5° to 38.5°. The magnesium concentration in the magnesium glycinate is considered to further increase when having one or more selected from the third peak and the fourth peak.

The top of the third peak is preferably in a 2θ range of 19.7° to 20.3°, and more preferably in a 2θ range of 19.9° to 20.1° in a powder X-ray diffraction (XRD) spectrum. The top of the fourth peak is preferably in a 2θ range of 37.7° to 38.3°, and more preferably in a 2θ range of 37.9° to 38.1°.

In a more preferred embodiment, the magnesium glycinate of the present disclosure preferably further has one or more peaks selected from a fifth peak having a top in a 2θ range of 17.4° to 18.4°; a sixth peak having a top in a 2θ range of 18.1° to 19.1°; a seventh peak having a top in a 2θ range of 22.9° to 23.9°; and an eighth peak having a top in a 2θ range of 28.6° to 29.6° in a powder X-ray diffraction (XRD) spectrum. The magnesium concentration in the magnesium glycinate is considered to further increase when having one or more peaks selected from among the fifth to eighth peaks.

The top of the fifth peak is preferably present in a 2θ range of 17.6° to 18.2°, and more preferably in a 2θ range of 17.8° to 18.0°. The top of the sixth peak is preferably present in a 2θ range of 18.3° to 18.9°, and more preferably in a 2θ range of 18.5° to 18.7°. The top of the seventh peak is preferably present in a 2θ range of 23.10 to 23.7°, and more preferably in a 2θ range of 23.3° to 23.5°. The top of the eighth peak is preferably present in a 2θ range of 28.8° to 29.4°, and more preferably in a 20 range of 29.0° to 29.2°.

In the magnesium glycinate of the present disclosure, the crystallite diameter of the crystal assigned to at least the first peak is preferably 100 Å to 800 Å, more preferably 200 Å to 600 Å, and still more preferably 250 Å to 500 Å.

In the present disclosure, the crystallite diameter D of magnesium glycinate may be calculated from the shape of the first peak in the XRD spectrum on the basis of the following formula:

$$D = K λ / B cos θ$$

wherein λ=1.5418 Å,
K=0.9,
B: FWHM, and
θ: peak top phase.

In the present disclosure, the XRD spectrum of magnesium glycinate may be measured by powder X-ray diffraction. In the powder X-ray diffraction measurement, a CuKα ray (λ=1.5418 Å) may be used as an X-ray source, an acceleration voltage may be 45 kV, and an angle step may be 0.026°.

The magnesium glycinate of the present disclosure preferably comprise a reduced amount of hydration water, and preferably is free of hydration water. The concentration of hydration water in the magnesium glycinate of the present disclosure may be preferably 0 wt % to 14 wt %, more preferably 0 wt % to 10 wt %, and still more preferably 0 wt % to 5 wt % based on the total amount of the magnesium glycinate. When the magnesium glycinate comprises a reduced amount of hydration water, the magnesium concentration in the magnesium glycinate may further increase.

The amount of hydration water in the magnesium glycinate of the present disclosure may be measured by thermogravimetric analysis (TG). Specifically, the amount of hydration water may be measured as the difference between the weight at 180° C. and the weight at 50° C. when thermogravimetric analysis is performed on magnesium glycinate at a heating rate of 10° C./min. The concentration of hydration water in the magnesium glycinate of the present disclosure can be calculated as a value obtained by dividing the amount of hydration water calculated as described above by the weight at 50° C. in the thermogravimetric analysis.

More specifically, in thermogravimetry (TG)-differential thermal scanning calorimetry (DSC) measurement at a heating rate of 10° C./min, a temperature width is defined by an onset temperature and an end temperature of an endothermic peak with an onset temperature of about 125 to 160° C. and an enthalpy of about 0 to 600 J/g on DSC data, and the amount of hydration water can be calculated on the basis of a weight difference in the thermogravimetric analysis in the temperature width.

In the present disclosure, the onset temperature of the peak is a temperature at an intersection of a straight line obtained by extending the baseline on the low temperature side of the peak toward the high temperature side in the DSC curve and a tangent drawn at a point where the gradient is maximum in the curve on the low temperature side of the peak. In addition, the end temperature of the peak is a temperature at an intersection of a straight line obtained by extending the base line of the peak toward the low temperature side in the DSC curve and a tangent drawn at a point where the gradient is maximum in the curve on the high temperature side of the peak.

The magnesium glycinate of the present disclosure may have a rate of weight loss at 380° C. of preferably 0 wt % to 30 wt %, more preferably 5 wt % to 25 wt %, and still more preferably 10 wt % to 22 wt % based on the weight at 50° C. in thermogravimetric analysis. The weight loss in the range of 50° C. to 380° C. is considered to be derived from hydration water in the magnesium glycinate, combustion of glycine present as an impurity in the magnesium glycinate, and thermal decomposition of the magnesium glycinate as a compound. Suppressing such weight loss is considered to further increase the concentration of magnesium in the magnesium glycinate.

In the magnesium glycinate of the present disclosure, the minimum temperature at which the rate of weight loss is 5 wt % or more based on the weight at 300° C. (hereinafter, also referred to as “weight loss onset temperature”) in thermogravimetric analysis is preferably 350° C. or more, more preferably 350° C. or more and 370° C. or less, and still more preferably 350° C. or more and 365° C. or less. The weight loss in the temperature range of 300° C. or more is considered to be derived from thermal decomposition of magnesium glycinate, and when the weight loss onset temperature is 350° C. or more, the purity of magnesium glycinate is considered to be higher, and the concentration of magnesium in the magnesium glycinate is considered to further increase.

In the present disclosure, thermogravimetric analysis may be performed at a heating rate of 10° C./min in a range of 30° C. to 600° C. under a nitrogen atmosphere or an air atmosphere.

In the magnesium glycinate, in differential scanning calorimetric analysis, a value S₁/S₂ obtained by dividing an area S₁ of an endothermic peak having an onset temperature in a range of 125° C. to 160° C. by an area S₂ of an endothermic peak having an onset temperature in a range of 330° C. to 370° C. is preferably 0 to 1.8, more preferably 0 to 1.5, and still more preferably 0 to 1.1. The endothermic peak having an onset temperature in the range of 125° C. to 160° C. is considered to be derived from the elimination of hydration water. In addition, the endothermic peak having an onset temperature in the range of 330° C. to 370° C. is considered to be derived from the melting of the magnesium glycinate. When the value S₁/S₂ is in such a range, the amount of the hydration water of the magnesium glycinate is considered to reduce, so that the concentration of magnesium may further increase.

The magnesium glycinate of the present disclosure preferably has an endothermic peak in a range of preferably 350° C. to 380° C., and more preferably 360° C. to 380° C. in differential scanning calorimetric analysis. The endothermic peak in the above range is considered to be derived from the melting of the magnesium glycinate.

The differential scanning calorimetric analysis may be performed at a heating rate of 10° C./min in a range of 40° C. to 600° C. under a nitrogen atmosphere using a simultaneous differential scanning calorimetric-thermogravimetric measurement type device.

In the magnesium glycinate of the present disclosure, for example, the concentration of Mg²⁺ in a saturated aqueous solution of magnesium glycinate at 25° C. may be preferably 1 g/L to 5 g/L, more preferably 2.2 g/L to 4.5 g/L, still more preferably 2.5 g/L to 4 g/L, and particularly preferably 2.8 g/L to 3.5 g/L.

The magnesium glycinate of the present disclosure has a high magnesium concentration, and for example, the content of magnesium in the magnesium glycinate at 25° C. may be preferably 12 wt % or more, more preferably 12.5 wt % to 20 wt %, and still more preferably 13 wt % to 17 wt %.

The concentration of Mg²⁺ in the saturated aqueous solution of magnesium glycinate at 25° C. can be measured by, for example, ion chromatography. The magnesium content may be calculated, for example, from the result of thermogravimetric analysis.

In a preferred embodiment, the magnesium glycinate of the present disclosure preferably comprise a first crystal grain having the first peak and the second peak in an XRD spectrum. Preferably, the first crystal grain has one or more peaks selected from the third peak and the fourth peak in the XRD spectrum, and more preferably has one or more peaks selected from among the fifth to eighth peaks.

Here, the content of crystal grains can be calculated using the following method. Firstly, the intensities of the diffraction pattern of a pure type I crystal and the diffraction pattern of a pure type II crystal are obtained, and their linear combination I_{c} is calculated by the following equation for all 2θ of 5°<2θ<30°.

$${I}_{c} ( 2 θ ) = a \times ( diffraction pattern intensity of type II crystal at 2 θ ) + b \times ( diffraction pattern intensity of type I crystal at 2 θ ) \\ 0 \leq a \leq 1 \\ 0 \leq b \leq 1 \\ a + b = 1$$

It is noted that the initial values of a and b are both 0.5.

Secondly, from the diffraction pattern intensity (I) of the sample for which the first crystal grain content is to be measured and the calculated pattern intensity I_{c}, the sum of squares J of residuals is calculated by the following equation.

$$J = {\sum }_{θ = {θ}_{0}}^{θ end} {( {I}_{c} - I )}^{2} {θ}_{0} : 5 ° \\ {θ}_{end} : 30 ° \qquad [ Mathematics equation l ]$$

Thirdly, with a and b as variables, a+b=1, 0<a<1, and 0<b<1 as constraints, a combination of a and b that minimizes the value of J is calculated, and the obtained value of a is taken as the content of the first crystal grain.

In the magnesium glycinate of the present disclosure, the content of the first crystal grain may be preferably 5 wt % to 100 wt %, more preferably 50 wt % to 100 wt %, still more preferably 80 wt % to 100 wt %, and particularly preferably 95 wt % to 100 wt %.

The magnesium glycinate of the present disclosure may comprise, in addition to the first crystal grain, a second crystal grain having a structure different from that of the first crystal grain. Examples of the second crystal grain include a crystal having a maximum peak in a 2θ range of 150 or more in an XRD spectrum. The content of the second crystal grain may be preferably 0 wt % to 30 wt %, more preferably 0 wt % to 10 wt %, and still more preferably 0 wt % to 5 wt %, based on the total amount of the crystals of the magnesium glycinate.

In the magnesium glycinate of the present disclosure, the intensity of the first peak or the second peak is preferably higher than the intensity of the maximum peak among the peaks having a 2θ in a range of 150 or more.

The magnesium glycinate of the present disclosure may further comprise magnesium hydroxide. The content of magnesium hydroxide may be preferably 0 wt % to 5 wt %, more preferably 0 wt % to 3 wt %, and still more preferably 0 wt % to 1 wt % based on the total amount of the magnesium glycinate.

The content of magnesium hydroxide in the magnesium glycinate may be quantitatively analyzed by using a least squares method, a reference intensity ratio (RIR) method, or the like in X-ray powder diffraction measurement.

The magnesium glycinate of the present disclosure has a color difference ΔE* measured by the following method that is preferably 30 or less, more preferably 25 or less, and even more preferably 20 or less. The lower limit of the color difference ΔE* may be 0 or more, may be 10 or more, and may be 15 or more. The magnesium glycinate of the present disclosure exhibits suppressed color change due to heat and exhibits good stability.

### [Method for Measuring Color Difference]

A mixture is obtained by mixing 1462 parts by mass of glucose and 500 parts by mass of magnesium glycinate and storing the mixture at 60° C. for 72 hours under a sealed condition. A color difference, ΔE*, relative to a reference point (L₀*=100, a₀*=0, b₀*=0) in an L*a*b* color system at a 10° viewing angle under D65 light source is calculated using an L* value, L*₁, an a* value, a*₁, and a b*value b*₁, of the mixture, according to the following formula.

$$Δ {E}^{*} = {[ {( {L}_{1}^{*} - {L}_{0}^{*} )}^{2} + {( {a}_{1}^{*} - {a}_{0}^{*} )}^{2} + ( {b}_{1}^{*} - {b}_{0}^{*} ) 2 ]}^{1 / 2}$$

Glucose and glycine may generate coloring components through the Maillard reaction. The magnesium glycinate of the present disclosure has a low glycine content (specifically, glycine that is not bound to magnesium and exists independently) and is considered to exist in a state where glycine and magnesium are stably bonded within the magnesium glycinate crystal. As a result, the Maillard reaction is suppressed, and it is considered to exhibit superior stability against heating.

As the color difference ΔE*, L*, a*, and b* values normalized to the CIE 1976 L*a*b* color space coordinates may be adopted and the values may be measured in accordance with JIS Z 8722. These measurements are performed using a D65 light source with a 10° viewing angle.

The average particle size of the magnesium glycinate of the present disclosure is preferably 20 μm to 350 μm, more preferably 25 μm to 300 μm, still more preferably 30 μm to 250 μm, and particularly preferably 30 μm to 100 μm. In the present disclosure, the average particle size means a volume-based median diameter (D50), and can be measured by a laser diffraction/scattering method.

In the magnesium glycinate of the present disclosure, the content of magnesium glycinate as a compound is preferably 90 wt % to 100 wt %, more preferably 95 wt % to 100 wt %, and still more preferably 97 wt % to 100 wt %.

The content of magnesium glycinate as a compound in the magnesium glycinate can be calculated by thermogravimetric analysis.

The tensile strength of the magnesium glycinate tablets is preferably 75 N/cm² to 500 N/cm²; more preferably 100 N/cm² to 400 N/cm²; and further preferably 100 N/cm² to 350 N/cm² when the magnesium glycinate is compressed using a flat punch (without roundness) at a compression pressure of 5 kN per tablet. Maintaining the tensile strength of the magnesium glycinate tablet within this range may facilitate stable tablet maintenance.

In this disclosure, “flat” means that the ratio of the curvature radius of the tablet to the diameter of the tablet (curvature radius/diameter) is 1.1 or more, more preferably 1.3 or more, and most preferably 1.4 or more. It should be noted that the curvature radius of a flat surface is infinite.

Magnesium glycinate may comprise optional magnesium compound in addition to magnesium glycinate as a compound. Magnesium oxide is an example of such an optional magnesium compound. The inclusion of such optional magnesium compounds is expected to increase magnesium content and improve flowability. The content of the magnesium compound is preferably 0 wt % to 25 wt %, more preferably 0 wt % to 20 wt %, and even more preferably 0 wt % to 15 wt %. When the magnesium compound content falls within this range, it may exhibit good compressibility, facilitating tablet formation.

### The Second Embodiment: The Production Method of Magnesium Glycinate

The magnesium glycinate of the present disclosure can be produced by a production method comprising:
a first step of mixing glycine and magnesium hydroxide in the presence of water to obtain a mixture; and
a second step of further mixing the mixture with ethanol to obtain magnesium glycinate.

The mixture may be either in a dissolved state or in a slurry state. From the viewpoint of production cost, a slurry state is preferred. The state of the mixture can be adjusted by the amount of water.

According to the above production method, magnesium glycinate having a high magnesium concentration may be produced. In addition, since the production method of the present disclosure does not use a component that may adversely affect the living body, the resulting magnesium glycinate is preferably used as a food and drink product and a pharmaceutical product. The present disclosure should not be construed as being limited to a particular theory, but the reason why such an effect is exhibited by the production method of the present disclosure is considered as follows.

That is, in the production method of the present disclosure, firstly, glycine and magnesium hydroxide are mixed in the presence of water, and then ethanol is further mixed. Therefore, it is considered that when glycine and magnesium are chelated to form magnesium glycinate, the intermolecular interaction between water and magnesium glycinate is changed by subsequently added ethanol, and magnesium cannot any longer capture a water molecule as a ligand, so that magnesium glycinate free of hydration water is generated. It is considered that as a result, magnesium glycinate having an increased magnesium concentration is obtained.

In the first step, glycine and magnesium hydroxide are mixed in the presence of water to afford a mixture. The chelation between glycine and magnesium hydroxide is considered to be promoted by such an operation.

Mixing order of water, glycine, and magnesium hydroxide is not limited, and water and glycine may be mixed first, and then the mixture may be further mixed with magnesium hydroxide; or water and magnesium hydroxide may be mixed first, and then the mixture may be further mixed with glycine. Alternatively, water, glycine, and magnesium hydroxide may be mixed at a time.

In one embodiment, it is preferable that water and glycine are mixed first, and then the mixture is further mixed with magnesium hydroxide. According to this embodiment, when an aqueous glycine solution acts on magnesium, magnesium glycinate in a dissolved state is considered to be generated, and anhydrous magnesium glycinate is considered to be generated as the magnesium glycinate is precipitated as a solid. When magnesium glycinate anhydride is generated as a nucleus, crystal growth of anhydrous magnesium glycinate is considered to proceed depending on temperature conditions. In addition, according to such an embodiment, the chelation between glycine and magnesium is considered to be promoted, and magnesium glycinate having a reduced amount of glycine may be more easily produced.

The amount of glycine is preferably 0.32 parts by weight to 7.7 parts by weight, more preferably 0.64 parts by weight to 6.4 parts by weight, and still more preferably 1.3 parts by weight to 5.1 parts by weight per part by weight of magnesium hydroxide. When the amount of glycine is in the above range based on the amount of magnesium hydroxide, it is easy to increase the reaction efficiency and to further increase the magnesium concentration in the resulting magnesium glycinate.

The amount of water is not particularly limited, but is preferably 0 parts by weight to 10 parts by weight, more preferably 0.125 parts by weight to 8 parts by weight, and still more preferably 0.25 parts by weight to 6 parts by weight based on 100 parts by weight in total of glycine and magnesium hydroxide. When the amount of water is in the above range, it is expected that the reaction efficiency is improved.

The mixing of the mixture in the first step may be performed by, for example, a stirring treatment. As a stirring device, a stirrer, a mixer, or the like can be used.

The first step is preferably performed at a temperature of preferably 50° C. or less, more preferably 10° C. to 40° C., and still more preferably 15° C. to 35° C. It is expected that the reaction efficiency is enhanced by performing the mixing of glycine and magnesium hydroxide at the above temperature.

The pH of the mixture in the first step is preferably 4 to 11, more preferably 5 to 9, and still more preferably 6 to 10.5. When the pH of the mixture is in the above range, the reaction efficiency is expected to improve and magnesium glycinate having a high magnesium concentration may be obtained.

The mixing time in the first step is preferably 30 minutes or more and 50 hours or less, more preferably 50 minutes to 30 hours, and still more preferably 60 minutes to 24 hours. When the mixing time is in the above range, the reaction efficiency is expected to improve.

In the second step, the mixture is further mixed with ethanol to afford magnesium glycinate. Mixing with ethanol is considered to make it easy to obtain magnesium glycinate having a high magnesium concentration.

The amount of ethanol is preferably 10 parts by volume to 400 parts by volume, more preferably 20 parts by volume to 350 parts by volume, still more preferably 40 parts by volume to 300 parts by volume, and particularly preferably 70 parts by volume to 250 parts by volume based on 100 parts by volume of water. It is considered that when the mixing amount of ethanol is in the above range, the magnesium concentration in the resulting magnesium glycinate is further increased. The aforementioned “amount of ethanol” means the amount of ethanol before mixing with the water.

The mixing of the ethanol and the mixture in the second step may be performed by, for example, a stirring treatment. As a stirring device, a stirrer, a mixer, or the like can be used.

The resulting magnesium glycinate may be subjected to a drying treatment and/or a pulverization treatment.

The drying temperature in the drying treatment is preferably 50° C. to 300° C., more preferably 50° C. to 250° C., and still more preferably 50° C. to 200° C. The drying time is preferably 30 minutes to 5 hours, more preferably 50 minutes to 4 hours, and still more preferably 1 hour to 3 hours.

The drying can be performed using a hot air dryer, a microwave dryer, a rotary dryer, or the like.

The pulverization can be performed such that the average particle size of the magnesium glycinate is preferably 20 μm to 350 μm, more preferably 25 μm to 300 μm, still more preferably 30 μm to 250 μm, and particularly preferably 30 μm to 100 μm.

The pulverization may be performed using a fine pulverizer such as a roller mill, a jet mill, a high-speed rotary pulverizer, or a container-driven mill.

The magnesium glycinate disclosed herein may be produced by the above-described manufacturing method, but is not limited to that produced by said method.

### The Third Embodiment: Magnesium Glycinate Tablets

The scope of the present disclosure comprises a magnesium glycinate tablet. In one embodiment, the magnesium glycinate tablet of the present disclosure comprises magnesium glycinate of the present disclosure.

In a preferred embodiment, the magnesium glycinate tablet of the present disclosure has a tensile strength of 75 N/cm² to 500 N/cm² when compressed using a flat punch (without roundness) at a compression pressure of 5 kN per tablet.

Moreover, the magnesium glycinate tablet of this embodiment may have a tensile strength of 75 N/cm² to 500 N/cm² when compressed using a flat punch (without roundness) at a compression pressure of 5 kN per tablet. Compression using a punch of other shapes is also possible, in which case the tensile strength may fall outside the above range. Such magnesium glycinate tablets are also comprised within the scope of the present disclosure.

The magnesium glycinate of the present disclosure exhibits excellent compressibility, and a tablet with a high concentration of magnesium glycinate is expected to be obtained. Furthermore, the magnesium glycinate tablet of the present disclosure are expected to have high tensile strength, enabling stable maintenance of the tablets.

The content of magnesium glycinate as a compound in the magnesium glycinate tablet is preferably 20 mass % to 95 mass %, more preferably 50 mass % to 95 mass %, and even more preferably 66.7 mass % to 90 mass %.

The magnesium glycinate content in the magnesium glycinate tablet of the present disclosure is preferably 0.1 mass % to 90 mass %, more preferably 10 mass % to 80 mass %, and even more preferably 20 mass % to 70 mass %.

The magnesium glycinate tablet may comprise as an internal additive or an external additive, a disintegrant such as low-substituted hydroxypropyl cellulose, agar, sodium croscarmellose, partially alpha-modified starch, potato starch, corn starch, calcium carboxymethylcellulose, crospovidone, and sodium carboxymethyl starch; a binder such as crystalline cellulose, hydroxypropyl cellulose, hydroxypropyl methylcellulose, and pullulan; a lubricant such as sucrose fatty acid esters, glycerol fatty acid esters, sodium stearyl fumarate, talc, stearic acid and their salts (Mg, Ca salts); a flow modifier such as fine-grained silicon dioxide, and calcined shell calcium; a sweetener such as aspartame, acesulfame potassium, and sucralose; an excipient such as calcium hydrogen phosphate, sugar alcohols, monosaccharides, disaccharides, oligosaccharides, cellulose, cellulose derivatives, starch, starch derivatives, and starch decomposition products; in addition to magnesium glycinate. The magnesium glycinate tablet may further comprise other additives such as amino acids or their salts, yeast extracts, vitamins, minerals, functional ingredients, polyphenols, adhesives, colorants, pH adjusters, buffers, and antioxidants.

The tensile strength of the magnesium glycinate tablet when compressed using a flat punch (without roundness) at a compression pressure of 5 kN per tablet is preferably 75 N/cm² to 500 N/cm², more preferably 100 N/cm² to 400 N/cm², and further preferably 100 N/cm² to 350 N/cm². The tensile strength within the above range may facilitate maintaining the tablet stably.

The shape of the magnesium glycinate tablets is not particularly limited and may be, for example, standard R, double-step R, sugar-coated R, angular R, angular flat, rounded flat, triangular, oblong, or hexagonal. The top surface shape of the tablet is also not particularly limited and may be flat, standard R, double-step R, or sugar-coated R. When the tablet surface is flatter, the tensile strength tends to become higher.

The size of the magnesium glycinate tablets is not particularly limited. In one embodiment, the diameter of the magnesium glycinate tablets is preferably 5 mm to 14 mm, more preferably 5 mm to 10 mm, and even more preferably 5 mm to 9 mm. The thickness of the magnesium glycinate tablet is preferably 2 mm to 7 mm, 3 mm to 6.5 mm, or 3.5 mm to 6.5 mm. The weight per tablet of the magnesium glycinate tablet is preferably 50 mg to 1,000 mg, more preferably 70 mg to 800 mg, and even more preferably 90 mg to 600 mg.

The magnesium glycinate tablet may be manufactured by a method comprising: a step of mixing magnesium glycinate with an internal additive, an external additive, and other additives as necessary to obtain a mixed powder; and a step of compressing the obtained mixed powder to obtain a magnesium glycinate tablet. When compressing the mixed powder, the mixed powder may be dispersed in a dispersing medium such as water and spray-granulated, or the mixed powder may be granulated as granules. Furthermore, a coating may be added in a subsequent process for the purpose of coloring the appearance to enhance commercial value or imparting taste or scent.

The average particle size of the mixed powder may preferably be 100 μm to 850 μm, more preferably 150 μm to 500 μm, and even more preferably 150 μm to 300 μm.

The tableting method is not limited, but may typically be performed using a tablet press. For example, a compression pressure per tablet is not limited, but is preferably 2 kN to 20 kN, more preferably 3 kN to 18 kN, and even more preferably 4 kN to 16 kN. Although higher pressure increases hardness, it also increases the load on the punch and the machine itself, leading to repair costs. Therefore, it is preferable to use the lowest possible pressure.

### The Fourth Embodiment: Pharmaceuticals or Food and Beverages

The magnesium glycinate of the present disclosure has a high magnesium concentration, and may be suitably used for various applications. Examples of such applications include a food and drink product and a pharmaceutical product, and the present disclosure also comprises a food and drink product and a pharmaceutical product comprising magnesium glycinate. The food and drink product and the pharmaceutical product may be produced using the magnesium glycinate of the present disclosure.

Magnesium glycinate is considered to have an action of replenishing magnesium in the body by being absorbed into the body. The intake of magnesium is expected to produce an effect of, for example, increasing the melatonin level in the brain, a relaxation effect, a good sleep effect, and an effect of stabilizing the blood glucose level. Magnesium glycinate is highly absorbed into the body while having a suppressed laxative action as a side effect, and thus it may be preferably used as a food and drink product and a pharmaceutical product.

In the present disclosure, “food and drink product” refers to general foods and drinks, and refers to general foods such as health foods; functional-indicated foods; insurance functional foods such as, for example, foods for specified health uses and nutritive functional foods; and supplements. In addition, the “food and drink product” is not limited to those administered to humans, and encompasses, for example, livestock feeds administered to animals, and pet foods.

The form of the food and drink product is not particularly limited, and may be any of solid, semi-solid, and liquid. Specific forms include, for example, tablets, pills, capsules, solutions, pastes, syrups, powders, and granules.

Also, the “pharmaceutical product” may be administered to a subject in any form and may be administered orally or parenterally. Preferably, the pharmaceutical product may be administered orally.

Such a pharmaceutical product may be used for treatment of diseases such as magnesium deficiency, insomnia, and depression, and various symptoms associated therewith.

The dosage form of the pharmaceutical product is not particularly limited, and may be a solid preparation for oral use such as a tablet, a granule, a powder, or a capsule; a liquid preparation for oral use such as an oral liquid preparation or a syrup; a liquid preparation for parenteral use such as an injection.

When the magnesium glycinate of the present disclosure is used for a food and drink product, the magnesium glycinate may further comprise an additive such as an excipient, a coating agent, a binder, an extender, a disintegrant, a surfactant, a lubricant, a diluent, a dispersant, a buffer, an osmotic pressure regulator, a pH regulator, an emulsifier, a preservative, a stabilizer, an antioxidant, a colorant, an ultraviolet absorber, a humectant, a thickener, an activity enhancer, an anti-inflammatory agent, a bactericide, a taste adjusting agents, and a scent adjusting agents.

The food and drink product or pharmaceutical product comprising the magnesium glycinate of the present disclosure can be used by administering it to a subject.

The magnesium glycinate of the present disclosure is expected to have a high magnesium concentration and high bioabsorbability. Therefore, the magnesium glycinate of the present disclosure is preferably used as a food and drink product or a pharmaceutical product. The magnesium glycinate of the present disclosure is also preferably used in a method for producing a food and drink product or a pharmaceutical product.

### EXAMPLES

The present disclosure will be described more specifically with reference to the following examples, but the present disclosure is not limited to them.

### Test Example 1

19.7 g of glycine (special grade, manufactured by FUJIFILM Wako Pure Chemical Corporation) was added to 50 mL of ion-exchanged water, and the mixture was stirred in a water bath at room temperature (22 to 26° C.) for 5 minutes or more (As One, VOLTEGA, Power Stirrer VPS-160SD). Next, 5.3 g of magnesium oxide (manufactured by Kyowa Chemical Industry Co., Ltd.) was weighed, added to the prepared solution, and then stirred in a water bath at room temperature (22 to 26° C.) for 24 hours. The resulting slurry was subjected to suction filtration, and the reaction product remaining on the filter paper (ADVANTEC, FILTER PAPER, QUANTITATIVE ASHLESS, 5C, 55 mm) was collected. This was dried at 105° C. overnight (Tokyo Rikakikai Co., Ltd., forced air flow oven WFO-520) and then pulverized using a mortar and a pestle, affording 12.1 g of a powder.

### Test Example 2

Glycine and magnesium oxide were reacted in the same manner as in Test Example 1, and the resulting slurry was subjected to suction filtration, whereby a reaction product on a filter paper and a filtrate were separately obtained. Then, an appropriate amount of ion-exchanged water was added dropwise to the reaction product for the purpose of removing impurities adhering to the reaction product, and the washing liquid was collected as a filtrate by suction again (total collected amount as a filtrate was about 75 mL). To the filtrate, 150 mL of ethanol (special grade, FUJIFILM Wako Pure Chemical Corporation) was added, and the mixture was stirred in a water bath at room temperature (22 to 26° C.) for 30 minutes. The solution after stirring was subjected to suction filtration, affording a reaction product remaining on a filter paper. This was dried and pulverized in the same manner as in Test Example 1, affording 9.1 g of a powder.

### Test Example 3

19.7 g of glycine was added to 50 mL of ion-exchanged water, and the mixture was stirred in a water bath at 30° C. for 5 minutes or more. A reaction was started in the same manner as in Test Example 1 except that glycine was stirred at 30° C. for 5 minutes or more. Next, 5.3 g of magnesium oxide was weighed, added to the prepared solution, and the mixture was stirred in a water bath at 30° C. for 50 minutes, and further stirred in a water bath at room temperature (22 to 26° C.) for 30 minutes. The resulting slurry was subjected to suction filtration, and a reaction product remaining on a filter paper (standard) was collected. This was dried and pulverized in the same manner as in Test Example 1, affording 7.06 g of a powder.

### Test Example 4

Glycine and magnesium oxide were added to ion-exchanged water in the same manner as in Test Example 3, and the mixture was stirred in a water bath at 30° C. for 2 hours and then further stirred in a water bath at room temperature (22 to 26° C.) for 30 minutes. Thereafter, filtration, drying, and pulverization were performed in the same manner as in Test Example 1, affording 8.56 g of a powder.

### Test Example 5

39.4 g of glycine was added to 50 mL of ion-exchanged water, and the mixture was stirred in a water bath at 80° C. for 5 minutes or more (stirring conditions were the same as those in Test Example 1). Next, 10.6 g of magnesium oxide was weighed, added to the prepared solution, and the mixture was stirred in a water bath at 80° C. for 50 minutes, and then stirred in a water bath at room temperature (22 to 26° C.) for 30 minutes. Thereafter, filtration, drying, and pulverization were performed in the same manner as in Test Example 1, affording 34.32 g of a powder.

### Test Example 6

19.7 g of glycine was added to 50 mL of a 1 mol/L aqueous sodium hydroxide solution (FUJIFILM Wako Pure Chemical Corporation), and the mixture was stirred in a water bath at 30° C. for 5 minutes or more (stirring conditions were the same as those in Test Example 1). Next, 5.3 g of magnesium oxide was weighed, added to the prepared solution, and the mixture was stirred in a water bath at 30° C. for 2 hours, and then stirred in a water bath at room temperature (22 to 26° C.) for 30 minutes. Thereafter, filtration, drying, and pulverization were performed in the same manner as in Test Example 1, affording 9.1043 g of a powder.

### Test Example 7

18.0 g of glycine was added to 50 mL of ion-exchanged water, and the mixture was stirred in a water bath at room temperature (22 to 26° C.) for 5 minutes or more (stirring conditions are the same as those in Test Example 1). Next, 7.0 g of magnesium hydroxide (manufactured by Kyowa Chemical Industry Co., Ltd.) was weighed, added to the prepared solution, and then the mixture was stirred in a water bath for 7 hours. Thereafter, filtration, drying, and pulverization were performed in the same manner as in Test Example 1, affording 3.8234 g of a powder.

### Test Example 8

18.0 g of glycine was added to 50 mL of ion-exchanged water, and the mixture was stirred in a water bath at room temperature (22 to 26° C.) for 5 minutes or more (stirring conditions are the same as those in Test Example 1). Next, 7.0 g of magnesium hydroxide was weighed, added to the prepared solution, and the mixture was stirred in a water bath for 2 hours. Then 50 mL of ethanol (special grade, FUJIFILM Wako Pure Chemical Corporation) was added, and the mixture was stirred in a water bath at room temperature (22 to 26° C.) for 30 minutes. Thereafter, filtration, drying, and pulverization were performed in the same manner as in Test Example 1, affording 16.9 g of a powder.

### Test Example 9

An experiment was performed in the same manner as in Test Example 1 except that the amount of glycine was changed to 18.0 g and 5.3 g of magnesium oxide was changed to 7.0 g of magnesium hydroxide, whereby 6.7366 g of a dry pulverized product was obtained.

### Test Example 10

Glycine and magnesium hydroxide were reacted in the same manner as in Test Example 9, and the resulting slurry was subjected to suction filtration, whereby a reaction product on a filter paper and a filtrate (about 75 mL) were separately obtained. Ethanol was added to the filtrate in the same manner as in Test Example 2, and then filtration, drying, and pulverization were sequentially performed, affording 13.2883 g of a white powder.

### Test Examples 11 to 14

Glycine (special grade, manufactured by FUJIFILM Wako Pure Chemical Corporation), magnesium glycinate (manufactured by Actylis), magnesium hydroxide (manufactured by Kyowa Chemical Industry Co., Ltd.), and magnesium oxide (manufactured by Kyowa Chemical Industry Co., Ltd.) were each used as a sample in Test Example 11, in Test Example 12, in Test Example 13, and in Test Example 14, respectively.

The powders obtained in the above Test Examples were subjected to the following measurements.

### (Powder X-Ray Diffraction Measurement)

The measurement was performed using an X-ray diffractometer EMPYREAN (Malvern Panalytical). The measurement sample was filled into a circular sample holder manufactured by Malvern Panalytical without gaps using a sample preparation kit manufactured by Malvern Panalytical. Measurement conditions are as follows.
X-ray source: CuKα (λ=1.5418 Å)
Acceleration voltage: 45 kV
Step: 2θ=0.026°
Scanning speed: 0.656514°/s
Divergence slit: 0.5000°
Scattering slit: 8.0 mm
Soller slit: 0.04 rad
HighScore Plus (Malvern Panalytical) was used for the analysis of the obtained XRD diffraction pattern, peak detection and peak fitting were performed, the phase and FWHM of the peak top were calculated from the obtained peak, and the crystallite size was calculated using the following formula.

$$D = K λ / B cos θ \\ λ = 1.5418 Å \\ K = 0.9$$

B: FWHM
θ: Phase of peak top

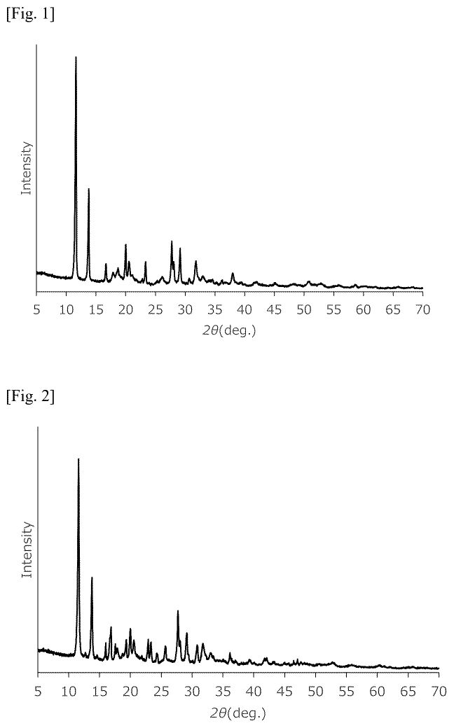

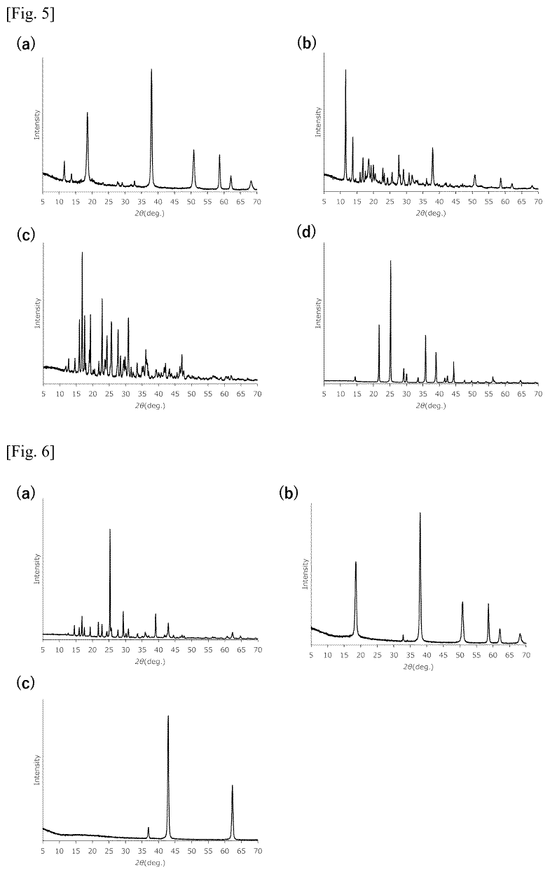

As shown in FIGS. 1 to 6, each of the magnesium glycinates obtained in Test Example 1 (FIG. 2), Test Example 3 (FIG. 4(b)), Test Example 6 (FIG. 4(d)), Test Example 8 (FIG. 1), and Test Example 9 (FIG. 5(b)) had a first peak having a top in a 20 range of 11.10 or more and 12.10 or less and a second peak having a top in a 2θ range of 13.3° or more and 14.3° or less, and it was confirmed that the magnesium glycinate was the magnesium glycinate of the present disclosure.

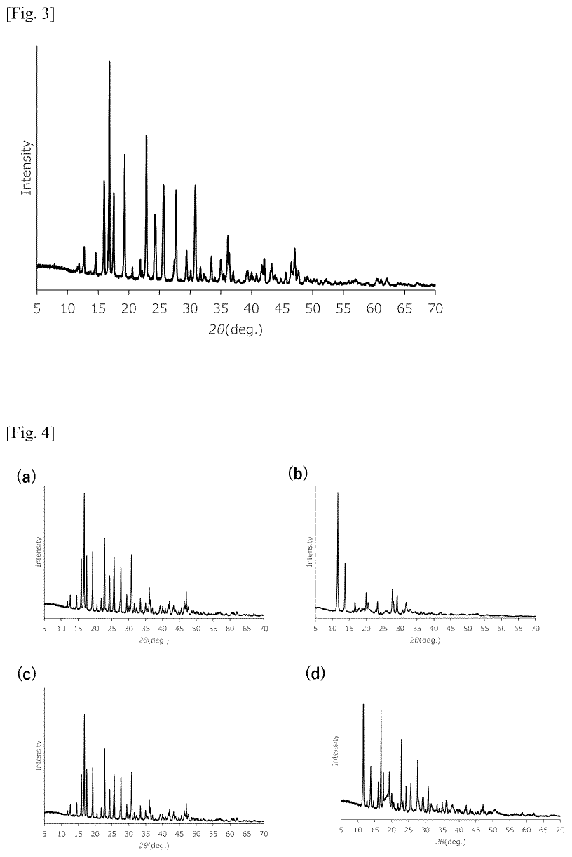

On the other hand, all of Test Example 2 (FIG. 4(a)), Test Example 4 (FIG. 3), Test Example 5 (FIG. 4(c)), Test Example 7 (FIG. 5(a)), Test Example 10 (FIG. 5(c)), and Test Example 12 (FIG. 6(a)) were confirmed to have neither a first peak having a top in a 2θ range of 11.10 or more and 12.10 or less nor a second peak having a top in a 20 range of 13.30 or more and 14.3° or less. In addition, Test Example 11 (FIG. 5(d)) is glycine, Test Example 13 (FIG. 6(b)) is magnesium hydroxide, and Test Example 14 (FIG. 6(c)) is magnesium oxide, and none of them contains magnesium glycinate.

### (XRD Measurement Result: Crystallite Size)

The crystallite size was calculated from the XRD diffraction pattern, and the results in Tables 1 and 2 were obtained. When the crystallite size of the peak corresponding to 16.7 to 17.0° and the crystallite size of the peak corresponding to 11.3 to 11.9° or 13.3 to 13.9° were compared, the crystallite size of the peak corresponding to 11.3 to 11.9° or 13.3 to 13.9° exhibited a smaller value. It is considered that a crystal that exhibits a peak corresponding to 11.3 to 11.90 or 13.3 to 13.9° is likely to be formed small.

TABLE 1

| Peak | Crystallite size (Å) | | | | |
|---|---|---|---|---|---|
| position | Test | Test | Test | Test | Test |
| (deg.) | exam- | exam- | exam- | exam- | exam- |
| — | ple 2 | ple 4 | ple 5 | ple 10 | ple 12 |
| 16.7-17.0 | 645.0433 | 665.1947 | 450.0441 | 575.5068 | 693.9151 |

TABLE 2

| | |
|---|---|
| Peak position | Crystallite size (Å) |

| (deg.) | Test | Test | Test |
|---|---|---|---|
| — | example 1 | example 3 | example 8 |
| 11.3-11.9 | 317.1010 | 414.7856 | 284.7628 |
| 13.3-13.9 | 357.1836 | 421.9333 | 324.2337 |

### (Simultaneous Differential Thermal Scanning Calorimetric-Thermogravimetric Analysis)

Measurement was performed using a differential thermal scanning calorimetric-thermogravimetric analyzer STA449 F3 Jupiter (NETZSCH-Geratebau GmbH). The measurement conditions are as follows.
Sample amount: 10 to 12 mg
Sample container: alumina pan
Heating rate: heating from 40° C. to 600° C. at 10° C./min
Atmospheric gas: nitrogen gas (70 mL/min)
Baseline measurement: empty pan

### (Thermogravimetric Analysis)

Measurement was performed using a thermogravimetric analyzer STA2500 Regulus (NETZSCH-Geratebau GmbH). The measurement conditions are as follows.
Sample amount: 10 to 11 mg
Sample container: aluminum pan
Heating rate: heating from 30° C. to 600° C. at 10° C./min
Atmospheric gas: nitrogen gas (50 mL/min) or air
Control substance: alumina (10 to 11 mg)

As shown in FIGS. 8, 10(a), 10(b), and 10(c), in Test Example 4, Test Example 6, Test Example 9, and Test Example 12 containing magnesium glycinate which exhibited neither the first peak nor the second peak, it was confirmed that weight loss occurred around 150° C. and in the range of 350° C. to 380° C. It is considered that the weight loss around 150° C. is derived from the elimination of hydration water from the magnesium glycinate and the weight loss in the range of 350° C. to 380° C. is derived from the thermal decomposition of the magnesium glycinate.

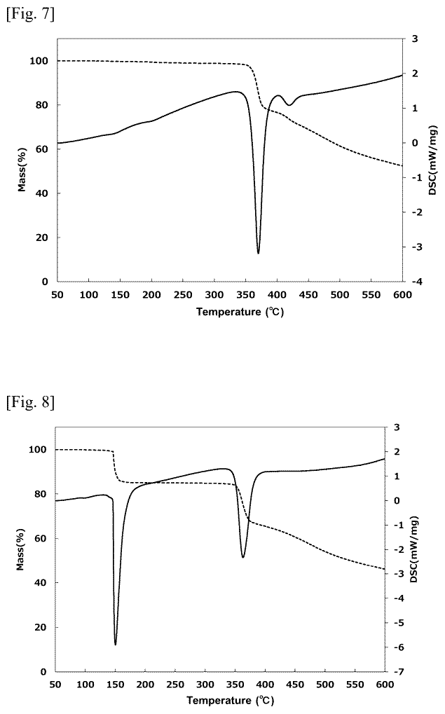

As shown in FIG. 7, in Test Example 8 containing the magnesium glycinate of the present disclosure, it was confirmed that no noticeable weight change occurred from 50° C. to around 350° C., and the weight decreased in the range of 350° C. to 380° C. From this fact, it is considered that the magnesium glycinate of Test Example 8 is free of hydration water.

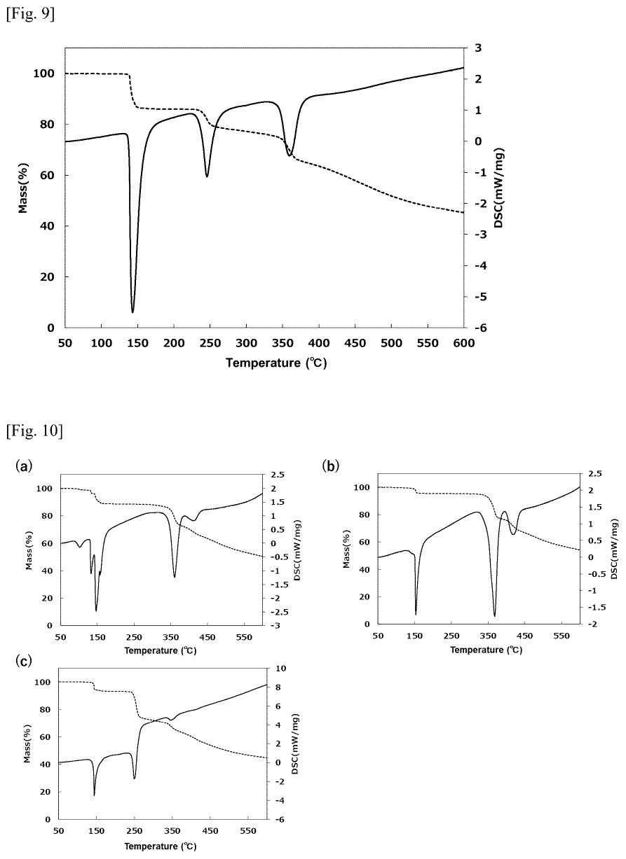

As shown in FIGS. 9 and 10(c), in Test Example 10 and Test Example 12, it was confirmed that weight loss occurred around 150° C., from 240° C. to around 268° C., and in the range of 350° C. to 380° C. The weight loss of from 240° C. to around 280° C. is considered to be derived from the combustion of glycine in the magnesium glycinate.

The results of thermogravimetric analysis of the samples of Test Examples 1, 2, 4, 6, 8, 9, and 10 to 12 are shown in Table 3.

When the content of hydration water was less than 0% by weight due to a measurement error or the like, the content was determined to be 0% by weight.

The content of glycine was calculated by dividing the difference between the weight at 280° C. and the weight at 240° C. by the weight at 50° C. in thermogravimetric analysis. The content of hydration water was calculated by dividing the difference between the weight at 180° C. and the weight at 50° C. by the weight at 50° C. in thermogravimetric analysis.

The content of magnesium glycinate was calculated by the following formula.

$$( Magnesium glycinate content ) = ( 1 - ( glycine content ) ) \times 100$$

The content of magnesium was calculated by the following formula.

$$( Magnesium content ) = ( magnesium glycinate content ) \times {M}_{A} / {M}_{B} \times 100$$

M_{A}: atomic weight of magnesium (24.31)
M_{B}: molecular weight of magnesium glycinate (Type I: 206.48 g/mol, Type II: 172.45 g/mol)

TABLE 3

| | | Test | Test | Test | Test | Test | Test |
|---|---|---|---|---|---|---|---|
| | | example | example | example | example | example | example |
| | | 1 | 2 | 4 | 6 | 8 | 9 |
| Content of glycine | wt % | 0.27 | 0.28 | 0.28 | 0.44 | 0.27 | 0.24 |
| Content of hydration | wt % | 11.7 | 18.0 | 14.8 | 11.2 | 0.4 | 4.8 |
| water | | | | | | | |
| Rate of weight loss at | wt % | 29.1 | 32.8 | 32.8 | 27.2 | 21.2 | 21.1 |
| 380° C. | | | | | | | |
| Weight loss onset | ° C. | 355.0 | 352.5 | 357.6 | 351.7 | 363.3 | 355.0 |
| temperature | | | | | | | |
| Content of magnesium | wt % | 99.73 | 99.72 | 99.72 | 99.56 | 99.63 | 99.76 |
| glycinate | | | | | | | |
| Content of magnesium | wt % | 11.74 | 11.74 | 11.74 | — | 14.04 | — |

| | | Test | Test | Test |
|---|---|---|---|---|
| | | example | example | example |
| | | 10 | 11 | 12 |
| Content of glycine | wt % | 13 | 100 | 45 |
| Content of hydration | wt % | 14.2 | 0.0 | 7.0 |
| water | | | | |
| Rate of weight loss at | wt % | 36.8 | 63.0 | 43.0 |
| 380° C. | | | | |
| Weight loss onset | ° C. | 350.0 | 325.0 | 335.0 |
| temperature | | | | |
| Content of magnesium | wt % | 87.35 | — | 54.94 |
| glycinate | | | | |
| Content of magnesium | wt % | 10.28 | — | 6.00 |

The results of the differential scanning calorimetric analysis of Test Examples 4, 6, 8 to 10, and 12 are shown in the following table.

The peak area S₁ (J/g) was calculated as the area of a peak having an onset temperature in the range of 125° C. to 160° C. The peak area S₂ (J/g) was calculated as the area of a peak having an onset temperature in the range of 330° C. to 370° C.

TABLE 4

| | Peak area (J/g) | | |
|---|---|---|---|
| | S₁ | S₂ | Peak area ratio S₁/S₂ |
| Test example 4 | 449.8 | 399.9 | 1.12 |
| Test example 6 | 276.1 | 350.1 | 0.79 |
| Test example 8 | 0.0 | 443.2 | 0.00 |
| Test example 9 | 122.1 | 381.3 | 0.32 |
| Test example 10 | 411.6 | 213.2 | 1.93 |
| Test example 12 | 276.5 | 44.65 | 6.19 |

### (Mass Spectrometry)

Measurement was performed using a mass spectrometer micrOTOF (Bruker Corporation). The measurement sample was dissolved in ultrapure water at 10 μg/mL, and the solution was ionized in an ESI Positive mode. The measurement was performed with the measurement mass range set to 50 to 1000 m/z, the capillary voltage set to 4500 V, and the nebulizer pressure set to 0.4 Bar.

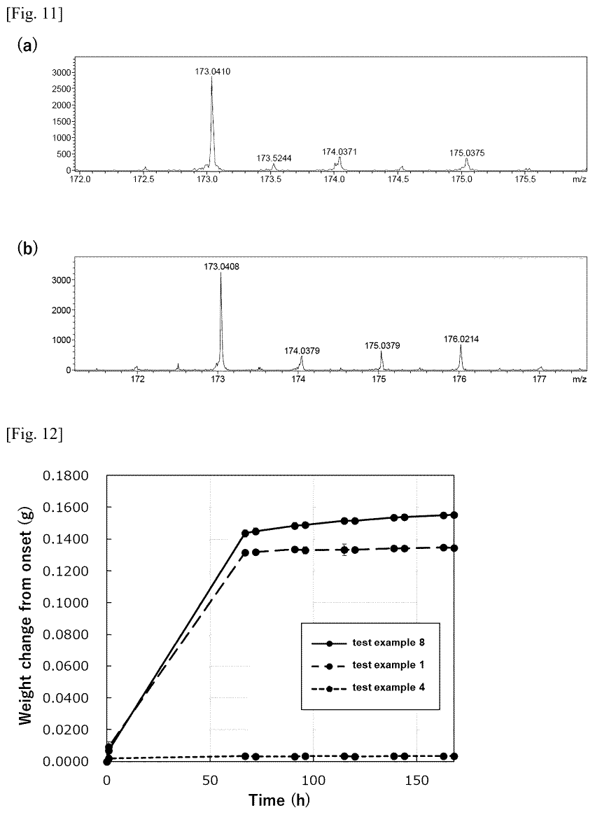

As a result of mass spectrometry, in both of Test Example 8 (FIG. 11(a)) and Test Example 4 (FIG. 11(b)), the compound in the obtained magnesium glycinate had a molecular weight of about 173.041 m/z, and it is suggested that magnesium glycinate as a compound was obtained.

### (Solubility Test)

1.5 g of the measurement sample was weighed, added to 50 mL of ion-exchanged water, and stirred at 500 rpm for 1 hour (AS ONE MAGNETIC STIRRER HSH-4D). The obtained slurry was subjected to suction filtration, and the filtrate was subjected to ultrasonic treatment (ultrasonic cleaner MCD-10, As One) and then filtered with a membrane filter (syringe filter DISMIC CS type, ADVANTEC). The obtained aqueous solution was diluted 500 times with ultrapure water, and ion chromatography analysis of magnesium ions was performed under the following conditions.

Ion chromatography of magnesium ions was measured using EcoIC (Metrohm). As a column, C6-150 (Metrohm) was used, and as an eluent, an aqueous solution of 1.7 mM nitric acid (Kanto Chemical Co., Inc.) and 1.7 mM dipicolinic acid (Kanto Chemical Co., Inc.) was prepared and used. Measurement was performed at a flow rate of 0.9 mL/min and an injection rate of 10 μL/sample. A 1002 g/L magnesium standard solution (Kanto Chemical Co., Inc.) was used as a magnesium ion standard solution, and a calibration curve of the concentration with respect to the peak area was calculated using a series of 100, 200, 400, and 800 times dilutions. The concentration of each sample was calculated from the area of the peak corresponding to magnesium obtained by ion chromatography, by using the calibration curve calculated above. The concentration of Mg²⁺ in the test solution in which each sample was dissolved was calculated using the molar mass of each ion and each molecule.

As a result, the concentration of Mg²⁺ in a test solution was 3.00 g/L in the sample of Test Example 8, 2.17 g/L in the sample of Test Example 4, and 2.19 g/L in the sample of Test Example 12. From this fact, it can be said that the sample of Test Example 8 has an increased magnesium concentration as compared with the samples of Test Examples 4 and 12.

### (Liquid NMR Measurement)

The measurement was performed using an NMR apparatus Avance III (¹H resonance frequency: 500 MHz) (Bruker Corporation). A 90° C. pulse was applied at 14 μs (¹H) and 9.1 μs (¹³C), and the signals were integrated until a sufficient signal-noise ratio was obtained. The sample was dissolved at 1 to 15 mg/mL in DMSO-d6 (FUJIFILM Wako Pure Chemical Corporation) or a 5% DSS/heavy water aqueous solution (FUJIFILM Wako Pure Chemical Corporation), and the solution was added to a sample tube having a diameter of 5 mm to perform ¹H, ¹³C, and HSQC-NMR measurements. Fourier transformation was performed using Bruker Top Spin software, affording NMR spectra.

Peaks detected by ¹H NMR measurement of glycine (Test Example 11) dissolved in heavy water appeared at 3.546 ppm and 4.771 ppm, and peaks detected by ¹³C NMR measurement appeared at 44.16 ppm and 175.11 ppm. Peaks detected by ¹H NMR measurement of type I magnesium glycinate (Test Example 4) dissolved in heavy water appeared at 3.306 ppm and 4.760 ppm, and peaks detected by ¹³C NMR measurement appeared at 45.83 ppm and 180.72 ppm. Peaks detected by ¹H NMR measurement of type II magnesium glycinate (Test Example 8) dissolved in heavy water appeared at 3.325 ppm and 4.780 ppm, and peaks detected by ¹³C NMR measurement appeared at 45.85 ppm and 180.99 ppm.

### (Hygroscopicity Test)

The sample was allowed to stand in a thermo-hygrostat (LH21-11M (Nagano Science)) for 7 days. The test conditions are as follows.
Temperature: 25° C.
Humidity: 60%
Sample weight: 0.9 to 1.1 g
Test duration: 7 days
Weight measurement: After 1 hour from start, after elapse of 3, 4, 5, 6, and 7 days

An increase in weight of 0.0018 g was detected when 1 hour had elapsed, 0.0035 g was detected when 3 days had elapsed, 0.0032 g was detected when 4 days had elapsed, 0.0035 g was detected when 5 days had elapsed, 0.0034 g was detected when 6 days had elapsed, and 0.0034 g was detected when 7 days had elapsed with respect to 1.01 g on average of the sample obtained in Test Example 4.

An increase in weight of 0.0067 g was detected when 1 hour had elapsed, 0.14 g was detected when 3 days had elapsed, 0.14 g was detected when 4 days had elapsed, 0.015 g was detected when 5 days had elapsed, 0.15 g was detected when 6 days had elapsed, and 0.15 g was detected when 7 days had elapsed with respect to 1.01 g on average of the sample obtained in Test Example 6.

An increase in weight of 0.0092 g was detected when 1 hour had elapsed, 0.13 g was detected when 3 days had elapsed, 0.13 g was detected when 4 days had elapsed, 0.13 g was detected when 5 days had elapsed, 0.13 g was detected when 6 days had elapsed, and 0.13 g was detected when 7 days had elapsed with respect to 1.00 g on average of the sample obtained in Test Example 8.

On the other hand, as a result of the observation of the appearance, aggregation of particles was observed in the sample of Test Example 4 after an elapse of 3 days, whereas no change in appearance was observed until the seventh day in the samples of Test Examples 6 and 8.

### (Particle Size Measurement Method)

0.7 g of magnesium glycinate was mixed with 70 mL of a solvent, and the particle size of the resulting mixed liquid was measured. A sample to be measured was dispersed in a solvent, and pretreated using an ultrasonic homogenizer US-300AT (Nihonseiki Kaisha Ltd.). The particle size of the obtained sample dispersion was measured using a particle size distribution analyzer MT3000 (MicrotracBEL). The measurement conditions are as follows.
Ultrasonication: 15 μm or 40 μm, 3 min
Particle permeability: permeable
Particle shape: nonspherical
Particle refractive index: 1.57
Solvent: ethanol (FUJIFILM Wako Pure Chemical Corporation)
Solvent refractive index: 1.36

The results shown in Table 5 were obtained for the particle size measurement. In Test Example 4 and Test Example 8, when the ultrasonic treatment was performed, the particle size was detected to be smaller than that when the ultrasonic treatment was not performed. From this fact, it is considered that the samples of Test Example 4 and Test Example 8 are in a state where smaller primary particles are associated with each other.

TABLE 5

Particle size D50 (μm)

| Ultrasonic treatment | Test | Test | Test |
|---|---|---|---|
| — | example 4 | example 8 | example 12 |
| None | 193.9 | 72.34 | 156.6 |
| 15 μm, 3 min | 51.86 | 46.96 | 102.9 |
| 40 μm, 3 min | 35.03 | 28.59 | 134.6 |

### (Evaluation of Tablet Strength)

Magnesium glycinate from Test Examples 4, 8, and 12 were mixed with crystalline cellulose (Asahi Kasei Corporation, Theolas UF-F702), fine-grained silicon dioxide (Fuji Silicia Co., Ltd., Silopage 720), and calcium stearate (Taihei Chemical Co., Ltd.) to form a formulation per tablet as shown in Table 6. The ingredients were mixed so that the total weight was to 4.5 g, and tableting was performed the following day.

Tableting tests were performed using a TYPE-M static compressor (Maekawa Test Machine Co., Ltd.) equipped with a punch that has a 10 mm diameter and a round, flat-bottomed shape; and a corresponding die. Each die cavity was filled with 300 mg of the mixture and compressed. Compression pressures of 5 and 10 kN were applied, and each test was repeated three times.

TABLE 6

| | | Test | Test | Test |
|---|---|---|---|---|
| | | example 13 | example 14 | example 15 |
| | | The amount of ingredients per tablet | | |
| Magnesium | Test | 200 | | |
| glycinate | example 8 | | | |
| | Test | | 200 | |
| | example 4 | | | |
| | Test | | | 200 |
| | example 12 | | | |

| | | | |
|---|---|---|---|
| Crystalline cellulose | 94 | 94 | 94 |
| Fine-grained silicon dioxide | 3 | 3 | 3 |
| Calcium stearate | 3 | 3 | 3 |
| Total weight | 300 | 300 | 300 |

### (Tensile Strength Measurement)

The tablets manufactured in each test example were cylindrical. Their thickness [mm] in the direction sandwiching the circular face was measured using a commercially available general-purpose digital thickness gauge. Tablet hardness was measured using a tablet hardness tester (DR. SCHLEUNIGER MODEL 6D TABLET TESTER), applying force horizontally to the circular surface. Hardness [N] was measured, repeating the test three times for each sample. The obtained values were converted to tensile strength using the following formula.

$$Tensile strength [ N / {cm}^{2} ] = 2 \times Hardness [ N ] / ( π \times 0.1 cm \times Tablet thickness [ cm ] )$$

Tablets were manufactured to evaluate the compressibility of magnesium glycinate with different crystal forms. The results of their strength evaluation as tensile strength are shown in Table 7.

TABLE 7

| | Test exam- | Test exam- | Test exam- |
|---|---|---|---|
| | ple 13 | ple 14 | ple 15 |
| Magnesium glycinate used | Test exam- | Test exam- | Test exam- |
| | ple 8 | ple 4 | ple 12 |
| Compression pressure 5 kN | 140.7 | 49.4 | 72.3 |
| Compression pressure 10 kN | 312.7 | 95.8 | 175.7 |

When using the magnesium glycinate from Test example 8, which is a new crystal form, tensile strength significantly improved with increased compression pressure, demonstrating superiority over the conventional crystal form as a raw material powder. Generally, tensile strength of about 100 N/cm² is considered sufficient to withstand distribution, indicating that this new crystal form may serve as a suitable raw material for tablet manufacturing. On the other hand, tablets manufactured using magnesium glycinate from conventional hydrated forms (Test examples 4 and Test example 12) exhibited weak strength and were unsuitable for tableting.

### (Evaluation of Maillard Reactivity)

1462 mg of glucose and 500 mg of magnesium glycinate were weighed, placed in a glass bottle, and were shaken to mix after capping the bottle. As a positive control, 1462 mg of glucose and 305 mg of glycine were weighed, placed in a glass bottle, and were shaken to mix after capping the bottle. As negative controls, pure glucose and pure glycine were similarly placed in glass bottles. All samples were stored in a 60° C. drying oven for three days, and the degree of coloration was observed. Glucose (Fujifilm Wako Pure Chemical, Special Grade) and glycine (Fujifilm Wako Pure Chemical, Special Grade) were ground in a mortar before use in the test.

The molar ratio of 1462 mg glucose to 500 mg magnesium glycinate is considered to be 2:1. Furthermore, the molar ratio of 1462 mg glucose to 305 mg glycine is considered to be 2:1.

Subsequently, the samples were recovered and the degree of coloration based on reflected light was analyzed using a colorimeter (ZE6000, Nihon Denshoku Kogyo Co., Ltd.) with irradiation and light reception conditions conforming to JIS Z-8722. The L*a*b* color space under a D65 light source with a 10° viewing angle was adopted. The degree of coloration was compared by calculating the color difference (ΔE* value) relative to the reference point (L₀*=100, a₀*=0, b₀*=0).

$$Δ {E}^{*} = {[ {( {L}_{1}^{*} - {L}_{0}^{*} )}^{2} + {( {a}_{1}^{*} - {a}_{0}^{*} )}^{2} + ( {b}_{1}^{*} - {b}_{0}^{*} ) 2 ]}^{1 / 2}$$

TABLE 8

| | Observation result | ΔE* |
|---|---|---|
| Test example 4 | No coloration | 18.1 |
| Test example 8 | No coloration | 18.9 |
| Test example 12 | Brown | 51.6 |
| Glucose and glycine | Slightly brown | 32.5 |

After storing at 60° C. for three days, the positive control exhibited browning, confirming the browning reaction test was viable. Test example 8 (anhydrous type) exhibited no coloration, confirming its resistance to Maillard reaction. Test example 12, with a high glycine content, turned brown. Test example 8 (anhydrous type) and Test example 4 (hydrated type) exhibited no coloration and retained their white color, confirming their resistance to Maillard reaction. It should be noted that even when ΔE* is approximately 18, the color may appear as white due to the calculation method which is based on the theoretical white point as the origin point.

### INDUSTRIAL APPLICABILITY

The magnesium glycinate of the present disclosure is expected to have a high magnesium concentration and high bioabsorbability. Therefore, the magnesium glycinate of the present disclosure is preferably used for food and drink products and pharmaceutical products.

1. Magnesium glycinate having a first peak having a top in a 2θ range of 11.1° to 12.1°; and a second peak having a top in a 2θ range of 13.3° to 14.3° in a powder X-ray diffraction spectrum.

2. The magnesium glycinate according to claim 1, wherein a ratio I₁/I₂ of an intensity I₁ of the first peak to an intensity I₂ of the second peak is 2 to 2.5.

3. The magnesium glycinate according to claim 1, further having one or more peaks selected from a third peak having a top in a 2θ range of 19.5° to 20.5°; and a fourth peak having a top in a 2θ range of 37.5° to 38.5° in the powder X-ray diffraction spectrum.

4. The magnesium glycinate according to claim 1, further having one or more peaks selected from a fifth peak having a top in a 2θ range of 17.4° to 18.4°; a sixth peak having a top in a 2θ range of 18.1° to 19.1°; a seventh peak having a top in a 2θ range of 22.9° to 23.9°; and an eighth peak having a top in a 2θ range of 28.6° to 29.6° in the powder X-ray diffraction spectrum.

5. The magnesium glycinate according to claim 1, wherein a rate of weight loss at 380° C. based on the weight at 50° C. is 30 wt % or less in thermogravimetric analysis.

6. The magnesium glycinate according to claim 1, wherein a value of S₁/S₂ obtained by dividing an area S₁ of an endothermic peak having an onset temperature in a range of 125° C. to 160° C. by an area S₂ of an endothermic peak having an onset temperature in a range of 330° C. to 370° C. is 0 to 1.8 in differential thermal scanning calorimetric analysis.

7. The magnesium glycinate according to claim 1, wherein the magnesium glycinate is free of hydration water.

8. The magnesium glycinate according to claim 1, wherein the color difference ΔE* measured by the following method is 30 or less:
[Method for Measuring Color Difference]
A mixture is obtained by mixing 1462 parts by mass of glucose and 500 parts by mass of magnesium glycinate and storing the mixture at 60° C. for 72 hours under a sealed condition. A color difference, ΔE*, relative to a reference point (L₀*=100, a₀*=0, b₀*=0) in an L*a*b* color system at a 10* viewing angle under D65 alight source is calculated using an L* value, L*₁, an a* value, a*₁, and a b*value b*₁, of the mixture, according to the following formula;
$$Δ {E}^{*} = {[ {( {L}_{1}^{*} - {L}_{0}^{*} )}^{2} + {( {a}_{1}^{*} - {a}_{0}^{*} )}^{2} + ( {b}_{1}^{*} - {b}_{0}^{*} ) 2 ]}^{1 / 2} .$$

9. The magnesium glycinate according to claim 1, wherein the tensile strength when compressed using a flat punch at a compression pressure of 5 kN per tablet is 75 N/cm² to 500 N/cm².

10. A magnesium glycinate tablet comprising magnesium glycinate according to claim 1.

11. A food and drink product or a pharmaceutical product comprising the magnesium glycinate according to claim 1.

12. A method for producing a food and drink product or a pharmaceutical product, comprising using the magnesium glycinate according to claim 1.

13. A method for producing magnesium glycinate, comprising: a first step of mixing glycine and magnesium hydroxide in the presence of water to obtain a mixture; and
a second step of further mixing the mixture with ethanol to obtain magnesium glycinate.

14. The method for producing magnesium glycinate according to claim 13, wherein the first step is performed at a temperature of 50° C. or lower.
